(19) United States
(12) Patent Application Publication
(10) Pub. No.: US 2010/0310528 A1
(43) Pub. Date: Dec. 9, 2010

(54) Vector System for Site-Specific Integration

(75) Inventors: Joseph Tony Pembroke, Limerick (IE); Anna Piterina, Limerick (IE); Timothy O'Brien, Bushypark (IE); Barry McGrath, Parteen (IE); Padraig Strappe, Sydney (AU)

Correspondence Address: FOLEY & LARDNER LLP, 975 PAGE MILL ROAD, PALO ALTO, CA, 94304, US

(73) Assignees: University of Limerick; National University of Ireland, Galway

(21) Appl. No.: 12/691,681

(22) Filed: Jan. 21, 2010

(30) Foreign Application Priority Data
Jan. 23, 2009 (IE) 2009/0073

(51) Int. Cl. A61K 35/12; C12N 9/12; C12N 5/02; C12N 5/00; A61K 38/45; A61P 35/00; A61P 37/00; A61P 31/00; A61P 25/00; A61P 9/00

(52) U.S. Cl. 424/93.7; 435/194; 435/375; 435/325; 424/94.5

(57) ABSTRACT

The integration system comprises at least one vector capable of expressing at least one exogenous gene, the vector comprising a coding sequence for an attP gene region and a coding sequence for at least one therapeutic molecule and an integrase. The integration system has an application in bacterial metabolic engineering and in gene therapy.

### FIELD OF THE INVENTION

The current invention relates to the field of nucleic acid integration and gene delivery. More specifically, the current invention relates to specific and stable integration of nucleic acid molecules into a host cell.

### BACKGROUND TO THE INVENTION

A mobile genetic element (MGE) is a type of genetic material that can move around within the genome of an organism. Within the prokaryotic world, there exist a variety of mobile genetic elements (MGEs) amongst which include, plasmids, phages, ICEs (integrating chromosomal elements) and conjugative transposons. Such elements exhibit extreme site-specificity when integrating into their bacterial host genomes and commonly consist of genes that confer a particular phenotype that is advantageous to the host in a particular environment. The increasing number of completed microbial genomes publicly available has revealed that mobile genetic elements play critical roles in the evolution of microbial populations, including the dissemination of fitness traits, competitive traits, antibiotic resistance traits, pathogenicity and virulence properties, metabolic properties and xenobiotic degradation and symbiosis properties.

Central to the idea of MGEs as a major force of evolutionary change, is the ability of certain MGEs to integrate into their host chromosome, such that all progeny will acquire and express traits encoded by the MGEs. MGEs typically insert their DNA into their hosts via recombination between a portion of the MGE called the attachment site P (attP site) and a similar DNA sequence on the bacterial chromosome called the bacterial attachment site (attB site), generating an integrated DNA element flanked by recombinant attL and attR sites. This recombination reaction is catalysed by an MGE-encoded recombinase protein (or integrase) protein, of which there are three main classes including serine recombinases, tyrosine recombinases and DDE transposases.

R391 is a 89 kb mobile genetic element belonging to a family of bacterial integrative conjugative elements (ICE) known as the SXT/R391 family. Originally found in the South African Providencia rettgeri strain, R391 is a genetic mosaic of plasmid, transposon and phage-like sequences. It is highly related to the SXT element, homologs of which are increasingly being found in Vibrio cholera species associated with the 7^{th} pandemic and responsible for drug resistance spread. Sequencing of the R391 element revealed that it contains an integrase of the tyrosine recombinase class, an element encoded attP site and lacks the ability to self-replicate, suggesting that its biology is to integrate into its host. Related SXT/R391 family elements such as SXT, R997, R392, R748 and ICESpuPO1 encode similar integrase systems and sites. All appear to be mosaics with a core genetic scaffold and adaptive genes such as antibiotic resistance determinants integrated into a number of hotspots on the ICE's (Osario et al 2008). Where analysed these elements do not appear to have an autonomous replicative stage (Nugent 1981; Pembroke et al 1986) and exist integrated into the prfC gene in their enterobacterial hosts (Murphy and Pembroke 1999; McGrath et al 2004; Pembroke and McGrath 2005).

Integration of R391 and other family members R997, pMERPH and R705 has been shown to occur site-specifically in gram-negative bacteria such as E. coli, Salmonella, Enterobacter, Vibrio, Proteus and Pseudomonas, by virtue of the integrase catalysing recombination of the element encoded attP site and the bacterial host encoded attB site (McGrath et al 2004) in a similar manner to that of lambdoid phage (Nunes-Duby et al 1998). The attP and attB sites associated with R391 integration are unique 17 base pair sites (McGrath et al 2004). The 17 base pair attB site is located in the host prfC gene. Integration of the ICE initially truncates and inactivates the prfC gene. The element subsequently restores the function of the prfC gene by encoding a new promoter and N′ terminus of a hybrid prfC, which are genetically fused to the host's truncated prfC (McGrath et al 2004). Such a site-specific integration mechanism is unique to the SXT/R391 family, which all encode a highly homologous but unique integrase (McGrath et al 2006).

Genetic knockout studies have shown that the orf5 gene of R391, encoding the R391 integrase is responsible for integration. A second gene, namely orf4, in combination with the integrase, is responsible for excision. The product of orf4, a UV inducible recombinational directionality factor, is a small basic protein, which interacts with the integrase to catalyse excision, effectively reversing the process of integration. No accessory host factors, such as integration host factor, are known to be required for this reaction to occur. This is in contrast with other site-specific recombination systems catalysed by other tyrosine recombinases (such as that of λ phage), which do require accessory host factors in addition to the att sites and the integrase protein.

Gene therapy has the potential to treat a wide range of diseases including blood disorders and cancer. Still in its infancy, this approach involves the delivery of genetic material to a cell or tissue to facilitate the production of a therapeutic protein. The delivery of the genetic material is commonly facilitated by a viral or non-viral vector means. There are significant hurdles facing researchers at present in their quest to develop an efficient, safe and long lasting delivery mechanism for use in gene therapy. Currently used integration vectors for gene therapy applications are beset with problems relating to the random nature of the vector's integration into the host chromosome with related potential for insertional mutagenesis and oncogenesis in the host. Resultantly, there has been a proliferation in the amount of research directed to the development of a vector adapted for site-specific recombination/integration in a range of bacterial and/or eurkaryotic host systems.

WO2006065103 discloses a recombinant vector for site-specific integration. The vector in question comprises the MJ1 gene, which encodes an integrase from Enterococcus temperate bacteriophage FFC1. WO9419460 and WO0116345 describe a vector system for site-specific integration of foreign DNA into a host cell. The methods described in this instance make use of phage pLC3 integration functions and bacteriophage lambda integrase, respectively. A number of integrase proteins capable of directing position specific integration of a vector into a cell, in particular the Ty3 integrase, are described by AEP0830455.

US2004110293, CA2413175 and WO2004048584 all relate to a method of sequence specific recombination of DNA into a cell involving providing the cells with at least one DNA sequence comprising an attB, attP, attL or attR sequence together with a bacteriophage lambda integrase. WO9720038 discloses a vector encoding a fusion protein comprising a retroviral integrase catalytic domain COOH-terminally coupled to a DNA binding protein, which is capable of integrating DNA into a target molecule. A Listeria mediated integration vector is described by WO03092600. The integration vector includes a listeriophage U153 integrase gene and an attachment site. This vector integrates at the comK integration site and the tRNAAr9 integration site. WO0187936 utilises the plasmid genes from Micromonospora carbonacea var. africana pMLP1 to develop a vector, which integrates in a site-specific manner into actinomycete species. WO9907861 discloses a DNA fragment that can direct insertion of DNA site specifically into Mycobacterium species. The fragment in questions comprises the attachment site attP and the integrase gene of mycobacteriophage Ms6. WO0075342 discloses a plasmid vector having an integrase gene derived from a Retrovirus suitable for site directed integration. WO0306687 and WO0107572 disclose a genetically engineered phiC31-integrase gene and vectors encoding such a gene for use in a method of site directed integration.

The current inventors have developed a bacterial and eukaryotic integration vector system, which exploits the site-specific integration abilities of R391 and other members of the SXT/R391 family. The integration system of the current invention is a dual vector system, comprising a helper vector expressing a bacterial integrase and a delivery vector containing the coding sites for the attP region. The delivery vector also contains a multiple cloning site to allow for the insertion of a wide variety of recombinant genes into the delivery vector. Upon entry into a target cell, the integration system facilitates the site-specific integration and stabilisation of any recombinant gene contained within the delivery vector in a number of bacterial and eukaryotic hosts containing the specific attB target site. The integration reaction results in stable association of attached genes to the host chromosome, obviating the need for subsequent selection for maintenance, e.g. antibiotic administration. The employment of a dual vector system imparts several advantages on the current invention. A smaller quantity of foreign genetic material or DNA is integrated into the genome, which gives a much lower probability of other undesirable recombination events. Using the dual system means that the integrase gene itself is not integrated into the host genome. It has previously been reported by WO306687 that periodic expression of integrase can cause mutation and chromosomal aberrations when integrated into host eukaryotic cells. Site-specific integration will also stabilise the integrated genes from loss following outgrowth, compared to non-integrating vector delivery systems such as standard plasmid cloning vectors.

The system developed by the current inventors is unique and advantageous in that the attP site and attB target site have a unique 17 base pair sequence at their core. The integrase, which catalyses the integration, is specific for the 17 base pair core attP and attB sites. An added advantage of this system is that integration can be easily reversed by the subsequent expression of the R391 encoded orf4 gene product, a recombinational directionality factor, which integrates along with the bacterial integrase, resulting in excision of integrated vectors or elements effectively reversing the integration process. The R391 orf4 is a UV inducible gene.

Integration in eukaryotic hosts is also possible utilising the system of the current invention, based on many eukaryotic species containing exact copies of the equivalent attB site. Unlike many specific integration vectors the attB integration site occurs several times within the human genome. The helper vector of the current invention contains a humanised integrase under the control of the CMV promoter, capable of catalysing site-specific integration into eukaryotic cells. The system of the current invention thus has an application for site-specific gene delivery to human cells for human gene therapy and recombinant protein production systems.

### OBJECT OF THE INVENTION

It is an object of the current invention to provide an integration system for site-specific delivery of genetic material into a cell. It is a further object of the current invention to provide an integration system comprising an integration helper vector based on an expressed integrase and an integration delivery vector containing the coding sequence for the attP gene region. It is a further object of the invention to provide an integration vector containing a coding region for an integrase and the coding sequence for the attP gene region. The current integration system thus has an application in bacterial metabolic engineering and in gene therapy for a number of disease applications, including but not limited to cancer gene therapy.

### SUMMARY OF THE INVENTION

The current invention provides an integration system comprising:
(i) at least one vector capable of expressing at least one exogenous gene, the vector comprising a coding sequence for an attP gene region and a coding sequence for at least one therapeutic molecule.
(ii) an integrase.

In a preferred embodiment the integrase may be encoded on the at least one vector. In a further preferred embodiment the integrase is encoded on a second vector capable of expressing at least one exogenous gene. In a still further embodiment, the integrase is delivered via liposome delivery. The integrase may be any of the SXT/R391 family of integrases. Preferably, the integrase is humanised. Preferably, the integrase has the sequence selected from the group comprising SEQ ID No.1, SEQ ID No.2, SEQ ID No. 3 or SEQ ID No. 4 or sequences substantially similar thereto which also encode an integrase.

The therapeutic molecule may be a nucleic acid molecule. The therapeutic molecule may be a DNA, RNA or amino acid molecule. The RNA molecule may be an siRNA, microRNA or shRNA. Preferably, the therapeutic molecule is selected from the group comprising, NOS (nitric oxide synthase), iNOS (inducible NOS), eNOS (endothelial NOS), VEGF (vascular endothelial growth factor), EFNB2 (Ephrin-B2) and OPN (osteopontin).

The attP gene region may be an R391 attP gene region or may be an attP gene region from any of the SXT/R391 family of ICEs. Preferably, the attP gene region comprises at least the 17 base pair attP core region together with its upstream promoter and downstream sequence of codons. Preferably, the attP gene region has the sequence of SEQ ID No. 7 or a sequence substantially similar thereto.

In a preferred embodiment of the current invention, the at least one vector further comprises the coding sequence for the bacterial orf4 gene. The orf4 gene sequence may be an orf gene sequence from any SXT/R391 family. In a preferred embodiment the orf4 gene is UV inducible. Preferably, the orf4 protein has the sequence of SEQ ID No. 9 or a sequence substantially similar thereto.

In one particular arrangement of the current invention, the at least one vector is temperature sensitive. Preferably, the vector is or is derived from the pSC101 vector. This vector contains a temperature sensitive rep protein necessary to allow plasmid replication.

The integration of the current invention can be employed to deliver genetic material to a target cell. Genetic material is any material in which genetic information is stored, such as DNA, RNA or amino acid sequences. The target cell may be any cell that contains an attB attachment site. Preferably, the attB gene region has the sequence of SEQ ID No. 8 or a sequence substantially similar thereto. The target cell may be a eukaryotic cell.

In another aspect the current invention provides a host cell transformed with the integration system of the current invention.

In a preferred embodiment the current invention provides the use of the integration system or a host cell of the current invention in the manufacture of a medicament for the treatment of disease. The disease may be selected from the group comprising cancer, genetic disease, infectious diseases, immunological disorders, neurological disorders or cardiovascular disease wherein the cardiovascular disease includes but is not limited to myocardial infarction, coronary vascular disease, peripheral vascular disease, ischemia, cerebrovascular disease, heart failure.

Unless otherwise defined, all terms of scientific terminology used herein are intended to have the meanings commonly understood by those of skill in the art. In some cases, terms are defined herein for clarity and the inclusion of such definitions herein should not necessarily be construed to represent a substantial difference over what is generally understood in the art or intended to limit the scope of the invention in any way. The current invention will now be described with reference to the following examples and figures. It is to be understood that the following detailed description and accompanying figures, are exemplary and explanatory only and are intended to provide a further explanation of the present invention, as claimed and not to limit the scope of the invention in any way.

### BRIEF DESCRIPTION OF THE DRAWINGS

FIG. 1 Construction of the R391 integrase expressing helper vector pIntHis based on pProEX-HtB. The integrase gene was amplified using the primers containing the cloning sites sited in Table 2a for directional insertion.

FIG. 2 A variety of vectors containing the 17 bp attP site cloned into the pGemT vector were constructed ranging from 80 to 353 by spanning the attP site. These were subsequently cloned into the chloramphenicol resistant vector pBT. One vector containing 100 bp insert was termed pattR391 and was used in subsequent studies.

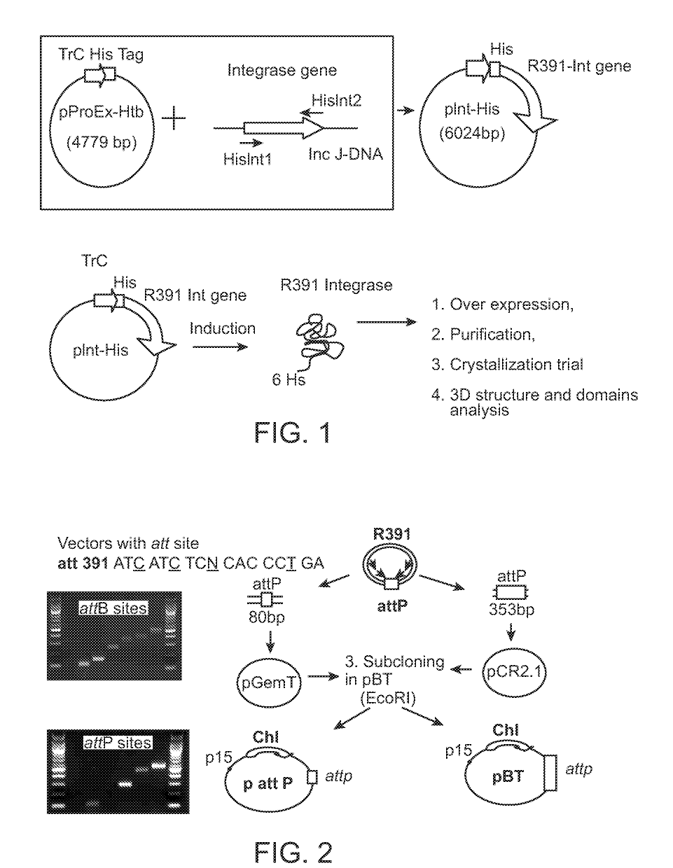

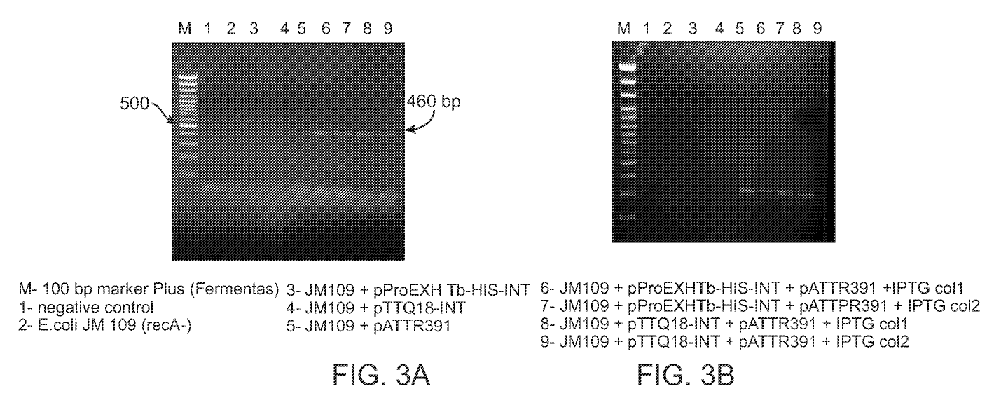

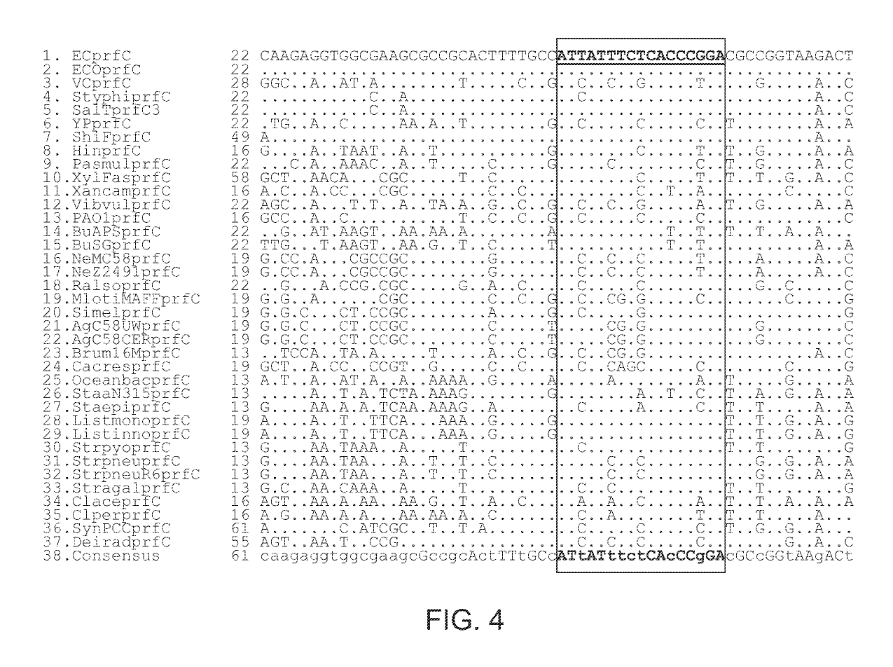

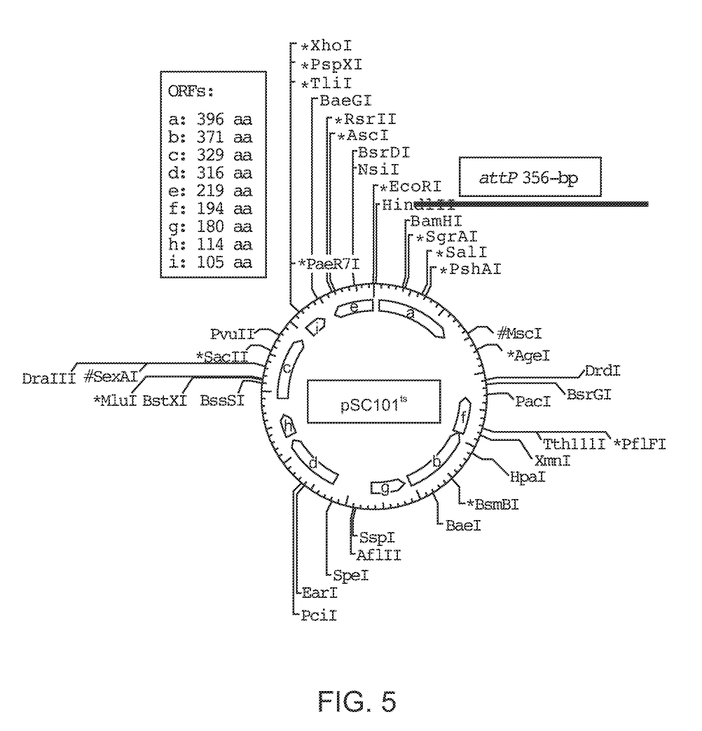

FIG. 3 Detection of site-specific integration of pattR391 following induction of integrase from pTTQ18-int and pProEXHtb-His int.
A) Represents PCR products generated using primers L1 and L2 which amplify the left integration junction with L1, this rev primer, being prfC specific to the left of the integration site and L2 the forward primer being vector specific
B) Illustrates PCR amplicons from primers R1 and R2 which amplify the Right integration junctions R1 being vector specific and R2 being specific to the prfC gene right of the integration site. In A and B Lanes M) Molecular weight markers Fermentas Plus, 1) negative control, 2) E. coli JM109 (recA) host), 3) JM109+pProEXHTb His int, 4) JM109+pTTQ18 int, 5) JM109+pattR391, 5) JM109+pProEXHTB His int+PattR391+IPTG 6) JM109+pattR391, 6) JM109+pProEXHTB His int+pattR391+IPTG, 7) JM109+pTTQ18 int His int+pattR391+IPTG, 8) JM109+pTTQ18 int His int+pattR391+IPTG

FIG. 4 Clustal W alignment of nucleotide sequences from bacterial species encoding prfC or prfC-like sequences. The complete gene sequences are not shown, only the 60 by region around the core 17 by R391 attachment site (attB) in E. coli K12 (line 1, highlighted in bold). The attB region is boxed to highlight similar sequences in other bacteria. The consensus sequence generated from this alignment is shown on line 38. Sequences were obtained from the following bacterial species (accession number of each locus in brackets): Line 1, Escherichia coli MG1655 (Z26313); 2, E. coli 0157:H7 (AE005668); 3, Vibrio cholerae (AE004152); 4, Salmonella enterica serovar Typhi (AL627284); 5, Salmonella enterica serovar Typhimurium LT2 (AE008914); 6, Yersinia pestis (AJ414142); 7, Shigella flexneri 2a str. 301 (AE015446); 8, Haemophilus influenzae Rd (U32846); 9, Pasteurella multocida PM70 (AE006120); 10, Xylella fastidiosa 9a5c (AE003871); 11, Xanthomonas campestris (AE012399); 12, Vibrio vulnificus CMCP6 (AE016802); 13, Pseudomonas aeruginosa PA01 (AE004807); 14, Buchnera sp. APS (AP001119); 15, Buchnera aphidicola Sg (AE014126); 16, Neisseria meningitidis MC58 (AE002418); 17, Neisseria meningitidis Z2491 (AL162754); 18, Ralstonia solanacearum GMI1000 (AL646086); 19, Mesorhizobium loti MAFF303099 (NC_{—}002678), 20, Sinorhizobium meliloti 1021(AL591783); 21, Agrobacterium tumefaciens C58 (University Washington) (AE009003); 22, Agrobacterium tumefaciens C58 (Cereon) (AE007970); 23, Brucella melitensis 16M (AE009662); 24 Caulobacter crescentus CB15 (AE005784); 25, Oceanobacillus iheyensis HTE831 (AP004600); 26, Staphylococcus aureus N315 (AP003132); 27, Staphylococcus epidermidis (AE016746); 28, Listeria monocytogenes EGD-e (A591977); 29, Listeria innocua CLIP 11262 (AL596167); 30, Streptococcus pyogenes SF370 (serotype Ml) (AE006578); 31, Streptococcus pneumoniae TIGR4 (AE007355); 32, Streptococcus pneumoniae R6 (AE008419); 33, Streptococcus agalactiae NEM316 (AL766847); 34, Clostridium acetobutylicum (AE007578); 35, Clostridium perfringens 13 (AP003187); 36, Synechocystis sp. PCC 6803 (D90906); 37, Deinococcus radiodurans R1 (AE001999).

FIG. 5 Map of pAP102. pAP102 contains the 356-bp attP amplicon from R391 cloned into pSC101^{ts} The map is based on the sequence of pSC101 (Bernardi and Bernardi1984). The 356-bp attP site was inserted into the EcoR1 site of pSC101^{ts}.

### DETAILED DESCRIPTION OF THE DRAWINGS

### Materials and Methods

### Bacterial Strains, Chemicals and Reagents

The bacterial strains used in this study are listed in Table 1. Bacterial strains were stored at −80° C. in LB broth containing 20% glycerol. All chemicals were obtained from Sigma (UK). All growth media was obtained from Oxoid (UK) unless otherwise stated. Media was supplemented with appropriate antibiotics obtained from Sigma (UK) and used at the following concentrations: 50 μg/ml⁻¹ ampicillin, 10 μg/m1⁻¹ chloramphenicol and 30 μg/ml⁻¹ kanamycin.

TABLE 1
Bacterial strains and vectors

| Bacteria and | | |
|---|---|---|
| vectors | Relevant to this study properties | Source |
| E. coli | | |
| AB1157 | Host of ICE element | This study |
| JM109 | General, maintaining plasmid | Promega, UK |
| DH5 α | Expression integrase | Invitrogen, UK |
| BL21 | Expression integrase | Invitrogen, UK |
| Plasmid and | | |
| ICE element | | |
| R391 | Source of integrase and attP site | This study |
| pGEM-T-easy | Amp, cloning attP site | Promega, UK |
| pBT | subcloning attP site in EcoRI site | Stratagene, UK |
| pTTQ18 | cloning integrase gene, expression of | Vector collection, |
| | native protein | Japan |
| pProEx-Htb | cloning, expression with 6His-Taq | Invitrogen, UK |
| pET-TOPO | cloning integrase gene, expression | Invitrogen, UK |
| | With His-Taq | |

### PCR and Sequencing

PCR and sequencing primers (Table 2) was synthesized by MWG-Biotech, Germany. Restriction enzymes were obtained from Roche Applied Bioscience (UK). PCR was carried out using Taq DNA polymerase (Bio-Line) in the buffer supplied by the manufacturer. The PCR program was as follows for the primer pair IntFor-Rev:94° C. for 4 min followed by 30-35 cycles of 94° C. for 1 min, 50-60° C. for 1 min and 72° C. for 1-3 min, followed by a final incubation at 72° C. for 3 min, hold at 4° C. Other PCR amplifications typically used the following conditions 95° C. for 10 mins; 80° C. for 2 mins, 95° C. 45s; 63.5° C. 45s; 72° C. 90s for 5 cycles, 95° C. 45s; 61.5° C. 45s; 72° C. 90s for 30 cycles, 72° C. 8 mins and 4° C. hold. Reaction conditions and electrophoretic manipulations are as described by McGrath and Pembroke 2004. All amplicons, such as integrase and attP and attB fragments were routinely sequenced using the relevant forward and reverse primers (MWG-Biotech, Ebersberg, Germany).

TABLE 2
(a) Primers used to generate recombinant constructs

| | | Restriction | |
|---|---|---|---|
| Name | Sequence (5′-3′) | sites | Application |
| Int-For | ACTGGATCCTAGGGCTGGGCTTATTAACA | BamHI | Cloning, |
| | TG | | plasmid |
| Int-Rev | TTAGCTATACGACTCTGCAGCGAAGA | PstI | pTTQl8 |
| IntHis-For | TCAGGATCCAACATGGCGTTATCAGTTAG | BamHI | Cloning, |
| | C- | | plasmid |
| IntHis-Rev | AGCCTCGAGATTTTACTGTCCGAAACGCC | XhoI | pProEx-Htb |
| | A | | |
| AttP-R391 | TGATAGCCGATTAGATAACC | TA-cloning | Cloning, |
| AttP-Rev- | ACACACTTTCCGAGGTTAC | sites | Plasmid |
| R391 | | | pGem-T-easy |
| RT-Int-For | CAATCAGCTAATAGCTGGCT | | Integrase |
| RT-Int-Rev | ACCGAGATGGGCTAAGTGCT | | mRNA |
| | | | detection |
| prfCCRE1 | TGATAGCCGATTAGATAACC | | Detection |
| prfCCLE1 | GTTTCTTCGTTGCACGAACTGG | | integration |
| | | | event |

TABLE 2
(b) Primers used to generate recombinant constructs

| | | | | | PCR product |
|---|---|---|---|---|---|
| | | | Dist | | size (and |
| | | Binds | from | | primer pair |
| Primer | Sequence | to | attB | Tm | to couple) |
| ABF1 | CTTATTTGCAAGAAGTGGCGAAGC | 716- | 38 (5′ | 62.7 | With RE1 |
| (For100) | | 739, | end), 15 | | (ABR1), = 113 |
| | | n = 24 | (3′end), | | bp |
| ABF2 | CGATGCCGCTTACTCAAGAAG | 674- | 80 | 62.3 | With RE1 |
| (For150) | | 694, | (5′end), | | (ABR1), = 155 |
| | | n = 21 | 60 from | | bp |
| | | | 3′end | | |
| ABF3 | GTTTCTTCGTTGCACGAACTGG | 568- | 186 | 60.25 | With RE1 |
| (LE1) | | 589, | (from | | (ABR1), = 262 |
| | | n = 22 | 5′end), | | bp |
| | | | 165 (3′) | | |
| ABF4 | CGATATTCTCGGTGGCATCAACG | 474- | 280 | 62.36 | (ABR3), = |
| (For500) | | 496, | (5′end), | | 542 bp |
| | | n = 23 | 258 | | |
| | | | (3′end) | | |
| ABR1 | CGGTCTGAATGGCCTGTCCGAA | 808- | 38 (from | 64.0 | As above |
| (RE1) | | 829, | 3′), 59 | | |
| | | n = 22 | (5′ end) | | |
| ABR2 | CGACTTAGCGTGCTGGTGGAAC | 851- | 81 (3′), | 64.4 | With |
| (Rev300) | | 873, | 103 (5′) | | LE1(ABF3) = |
| | | n = 23 | | | 305 bp |
| ABR3 | GGTGTCGAGCAGGTTAACCAGG | 969- | 199 (5′), | 63.94 | With LE1 |
| (Rev400) | | 948, | 178 93′) | | (ABF3) = 401 |
| | | n = 22 | | | bp |
| ABR4 | GCCGTCAGGTACGATAGGA | 1016- | 246 (5′), | 61.4 | With LE1 |
| (Rev500) | | 998, | | | (ABF3) = 448 |
| | | n = 20 | | | bp |

### Cloning and Genetic Manipulations

Genomic DNA extractions were performed as described by Sambrook et al 1989. Plasmid isolation was carried out using Promega™ plasmid isolation miniprep kits according to manufacturer's instructions. Competent cell was prepared as described by Chung et al, (1989). Transformation of E. coli was carried out by electroporation. Genetic manipulations and recombinant techniques used have been described by Spada et al 2001. Induction of cloned int^{R391} expression was carried using a final concentration of 1 mM IPTG. Reverse transcriptase reactions on the integrase gene were carried out to verify mRNA production from the integrase gene (O'Halloran et al 2007). Possible toxicity associated with over-expressed integrase was evaluated by analyzing any growth deviations in inducted cells.

The plasmid pProExHTb (Gibco-BRL), containing the His₆ tag with a TEV cleavage site under the control of an isopropyl-1-thio-β-d-galactopyranoside (IPTG) promoter of pTTQ (Japanese Plasmid Culture Collection) was used in cloning. Purified fragments were digested with appropriate restriction enzymes for directional cloning (see Table 2), ligated with T4 DNA ligase (Invitrogen) at 16° C. overnight to generate plasmid pProExHTb- or pTTQ-int. pGEM-T http://www.promega.com/tbs/tm042/tm042.pdf was and pBT http://www.stratagene.com/vectors/maps/pdf/pBT.pdf. were used to clone that attP site. Different primer sets (Table 2b) were utilised to optimise the attP fragment resulting in a number of different length amplicons. These were cloned into pBT. Transformants in all cloning experiments were selected in JM109 (recA), the general cloning procedures used have been described (Spada et al 2002).

The Ability of Cloned Integrase to Catalyse Site-Specific Integration of the attP Containing Delivery Vector

pBT attP was initially transformed into JM109, which was then transformed with pTTQ-int^{R391} or pProEXhtb-int^{R391}. Hosts containing both helper and delivery vectors were then induced with IPTG or left un-induced. Subsequently these cells were examined for integration of the delivery vector using PCR primers specific to the integrated form of the vector (Table 2a).

### UV Induction of the Circular Form of R391

Strains were incubated at 37° C. in nutrient broth (NB) to an optical density at 600 nm of 0.4. Cells were then harvested and resuspended in 50 ml of sterile M9 minimal media, and, after UV irradiation with a 15-W germicidal lamp emitting 16 ergs/mm² for 30 s, they were transferred to double-strength NB and incubated at 37° C. again for 20 min. Cells were collected and harvested for DNA extraction.

Construction of pAP44

A conditional integration vector based on the R391 integration system and the temperature conditional replicon pSC101^{ts} was also constructed. The tetracycline resistance plasmid, pSC101^{ts}, is able to replicate at 30° C. but unable to replicate at 42° C. (Gotah and Sekiguchi 1977). The 356-bp attP site of R391 was cloned into the vector to generate pAP44. This vector was then utilised as a delivery vector and its integration analysed by transforming hosts harbouring pAP44 with pTTQ-int^{r391} and observing integration.

### Results

The ability of a cloned integrase from the ICE R391, the prototype of the SXT/R391 family of ICE's, to catalyse integration of a vector containing the attP integration site of R391 site-specifically into the E. coli host chromosome in the absence of other R391 encoded functions was examined.

### Vector Construction and Analysis

The integrase of R391 was amplified using the primer pairs IntFor and IntRev, containing BamH1 and Pst1 and IntHisFor and IntHisRev containing BamH1 and Xho1 respectively (Table 2a). Amplicons were purified, sequenced to verify correctness and cloned into pProEx or pTTQ to give pProEXHtb-int^{R391} and pTTQ-int^{R391}. These vectors were subsequently re-sequenced and RT-PCR performed to ensure that integrase was expressed upon induction with IPTG. Sequence analysis revealed the correct sequence fused to the ptac promoters in both plasmid constructs. The constructs were then examined by RT-PCR upon induction with IPTG. In both cases over expression of the int^{R391} gene was observed. To determine if over expression had any toxic effect, the growth of JM109 with and without pProEXHtb-int^{R391} and pTTQ-int^{R391} and under induced and non-induced conditions was examined by monitoring growth rates comparatively over an 8-hour period. No difference in growth rates was observed indicating that over-expression was not apparently toxic to JM109.

To optimise the attP delivery vector a number of primer sets were utilised to amplify the attP site and surrounding sequences. Integration of R391 has been shown to require the 17-bp attP site but additionally an upstream promoter and a downstream sequence of codons, which upon integration are fused to the truncated prfC gene to generate a new hybrid and functional prfC gene (McGrath and Pembroke 2004). To optimise this construct a number of amplicons of different lengths were generated (FIG. 3), cloned and subsequently examined for integration. We observed that attP constructs of 200-bp were optimal and this amplicon was utilised to generate the delivery vector. attP amplicons were sequenced and cloned into pBT.

### Examination of Vector Integration.

The delivery vector pBT-attP was initially transformed into JM109 and examined for integration. This was analysed utilising a primer specific to the pBT-attP vector and another specific to the c-terminal region of the host prfC gene, which were designed to amplify the integration junction. Any un-catalysed integration event would allow amplification of this junction fragment however in this case no integration event was observed in the absence of the presence of int^{R391} (FIG. 3 lane 4) as judged by the absence of a junction amplicon. JM109 harbouring pBT-attP was subsequently transformed with pProEXHtb-int^{R391} and pTTQ-int^{R391} and the ability of these constructs to catalyse integration examined. Using the junction primers described, no integration was observed (FIG. 3 lanes2-6). Treatment of JM109 pBT-attP, pProEXhtb-int^{R391} and JM109 pBT-attP, pTTQ-int^{R391} with an inducing dose of IPTG was now shown to induce integration (FIG. 3 lanes 6-8). This integration was verified by the detection of the junction specific amplicons, which were subsequently sequenced from a number of different clones. Sequencing verified that integration of pBT-attP had occurred site specifically catalysed by the induced int gene from both helper vectors into the 17-bp attB site in the prfC gene in JM109. In the case of pProExhtb-int^{R391}, the addition of the his tag in this construct did not appear to effect integration.

### Stability of the Integrated Delivery Vector and Curing of the Helper Vector

Following integration a number of integrated delivery vector clones were chosen to analyse the stability of the integration event and the rate of helper vector curing following removal of selective pressure.

Cultures were grown in liquid media for 7 days with subculture into fresh non-selective media every 24 hours. It was noted that the helper vector was lost completely after 4 days subculture under non-selective conditions. No loss of the integrated delivery vector was observed in the absence of selection after 7 days indicating that the integration event was stable. This is not surprising since results show that integration of R391 and related SXT/R391 family elements tends to be extremely stable and are rarely lost upon months of subculture in the absence of selection (Pembroke unpublished). The number of integrated delivery vector containing hosts was also examined, after 7 days subculture via the junction assay described earlier and in all cases the amplicons were intact. This stability may reflect the nature of the integration locus such that deletions of the integration vector could lead to loss of the prfC gene, which encodes an essential factor for protein synthesis.

Integration of pAP102 (pSC101^{ts}-attP^{R391})

The 356-bp attP site from R391 was cloned into the EcoR1 site of pSC101^{ts} (FIG. 5) and clones analysed for the correct construct. PSC101 is a temperature sensitive vector. It contains a temperature sensitive rep protein necessary to allow plasmid replication and copying so that they plasmid can exist in a replicative state and exist separately to the bacterial chromosome. Use of this temperature sensitive derivative means that at low temperature the rep protein functions while at elevated or non-permissive temperatures the rep protein will not function. Thus at elevated temperatures the vector will not replicate and will be lost from the cell. If the vector contains an integrase attP site it will be forced to integrate at the non-permissive temperature to prevent it from being lost from the cell. Any genes encoded on the vector will therefore integrate into the attB site. One plasmid, termed pAP 102, was used for further study. HB101 (recA) cells harbouring pAP44 we found to retain the tetracycline resistance replicon at 30° C. but found to loose the determinant at 42° C. Analysis of the prfC gene in these strains revealed that the native prfC gene was intact indicating that no integration event had occurred. Transformation of pTTQ-int^{R391} into these cell followed by induction of the integrase revealed stable inheritance of the tetracycline resistance phenotype at the non-permissive temperature. PCR analysis of the prfC gene revealed that vector pAP102 had integrated site specifically in a similar manner to pBT-attP. These data indicated that vectors based on pSC101^{ts} could be utilised for site specific integration allowing the functionality of cloning sites from this vector for targeted delivery into the prfC gene.

### Integration of IncJ Integrase

The IncJ integrase was cloned into a eukaryotic expression vector, pCMVTag2a, to facilitate testing of gene expression in mammalian cell lines. Initial expression analysis by RT-PCR suggests that the IncJ integrase (both wild-type and synthetic) is expressed in certain mammalian cell lines after integration with the vector system of the current invention, including hMSCs. The minimal amount of IncJ DNA required for site-specific insertion was subcloned from pBT-ATTP into a eurkaryotic backbone vector, pCMVZeo, creating pCMVZeoattP. This construct contains an antibiotic (Zeocin) resistance gene, which enabled enrichment and selection of human cell lines with attP construct. Functional analysis of the IncJ-Int was performed by co-transfecting mammalian cell lines (hMSCs) with pCMV-Tag2-Int and pCMVZeoattP. Some Zeocin resistant hMSC colonies were identified suggesting that the system functions effectively in hMSCs.

Bioinformatic Analysis of attB Sites in Bacteria.

The prfC gene from some 37 bacterial hosts was analysed by aligning the prfC sequences and searching these for the consensus 17-bp attB integration sequence (McGrath and Pembroke 2004) common to ICE's of the SXT/R391 family (FIG. 4). In all cases analysed we observed a homologous if not identical 17-bp sequence in the strains analysed and in a similar position within that host's prfC gene. This raises the potential that SXT/R391 ICE's may integrate into a very wide range of bacterial hosts as the prfC sequence is widely conserved and raises the possibility that this vector delivery system may have wide ranging applicability for site-specific delivery into a wide range of hosts.

### Discussion

The current inventors have genetically manipulated members of the family of bacterial integrative conjugative elements (ICE), known as the SXT/R391 family, in particular the R391 genetic element, to develop an integration system capable of integrating foreign genetic material into a target cell, by means of an attP site and an expressed integrase. Site-specific integration, effected by the integrase, can occur into any host-encoded attB site. The delivery vector contains a multiple cloning site to allow for the insertion of a wide variety of recombinant genes or genetic material into the delivery vector. Upon entry into a target cell, the integration system facilitates the site-specific integration and stabilisation of any recombinant DNA or genetic material contained within the delivery vector in a number of bacterial and eukaryotic hosts containing the specific attB target site. The integration results in stable association of attached genes to the host chromosome and their subsequent expression in the host cell.

In a preferred embodiment, the integration system of the current invention consists of a dual vector system. The use of such a dual system imparts several advantages. A smaller quantity of foreign DNA is integrated into the genome, which gives a much lower probability of other undesirable recombination events. Using the dual system means that the integrase gene is encoded on a helper vector and as such is not integrated into the host genome. The integrase may also be delivery to target cell by liposome delivery. Again, using this method the integrase could only be present in the target cell transitorily and would be subsequently degraded. It has previously been reported by W0306687 that periodic long-term expression of integrase can cause mutation and chromosomal aberrations when integrated into host eukaryotic cells.

The current inventors have shown the ability of a cloned R391 integrase to catalyse integration of a delivery vector containing the attP integration site into an E. coli host chromosome. The integration was verified by subsequent sequencing from a number of different clones confirming that integration occurred site specifically into the 17 base pair attB site in the prfC gene in JM109 competent cells. Furthermore, the inventors confirmed the stability of the integration reaction. In one particular embodiment of the current invention the inventors developed and utilised a temperature sensitive delivery vector. Use of this temperature sensitive derivative means that at low temperatures the vector will replicate while at elevated or non-permissive temperatures it will not and will be lost from the cell. If the vector contains an integrase attP site it will be forced to integrate at the non-permissive temperature to prevent it from being lost from the cell. Any genes encoded on the vector will therefore integrate into the attB site.

The data presented indicate that the SXT/R391 family integrase encoded by the prototype element, R391 is the only element-encoded activity that is required to catalyse site-specific integration of the elements attP site and that integration occurs in a recA host. Although conjugation of ICEs in enterobacterial hosts has been reported to require integration host factor there appears to be no requirement for host-encoded factors such as FIS or IHF in integration (McLeod et al 2006). This would indicate that ICEs could thus transfer widely to a broad range of hosts in the natural environment as no host specific integration factors appear to be required for integration. Equally the observation here that integrase appears to be the only element encoded activity necessary for integration would suggest that the element would support the hypothesis of broad transferability provided the integrase was expressed, functional, and that the attB site was present. The current invention has several advantages for gene delivery over currently employed integration systems described in the literature. Presently used integrating vectors for gene therapy applications are beset with problems relating to the random nature of the vectors integration into the host chromosome with related potential for insertional mutagenesis and oncogenesis in the host. Furthermore, cloning via plasmid vectors necessitates maintenance of the recombinant vector via the use of selective antibiotics in the subsequent growth media. Integration via the current system results, however, in stable maintenance and no subsequent antibiotic selection is required. Site-specific integration stabilises the integrated genes from loss following outgrowth, compared to non-integrating vector delivery systems such as standard plasmid cloning vectors. An additional advantage is that the integration event may be reversed by expression of the ICE R391 encoded orf4 gene product. The use of this gene can provide a mechanism of reversing the integration event should this be required.

The human genome contains several attB-like sites located in non-coding regions of the genome. This raises the possibility of utilising the integration system of the current invention, for the delivery and integration of new target genes in gene therapy applications or metabolic engineering of eukaryotic cells, including human cells.

The results presented illustrate that the current inventors have developed a site-specific integration system capable of stably integrating any foreign genetic material into a target cell whether it be a eukaryotic or a prokaryotic cell. Circumventing the pitfalls of previously employed gene delivery systems the current integration system is ideal for use in bacterial metabolic engineering and in gene therapy applications including cancer gene therapy.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. In so far as any sequence disclosed herein differs from its counterpart in the attached sequence listing in PatentIn3.3 software, the sequences within this body of text are to be considered as the correct version.

### SEQ IDs

Sites of probes, oligonucleotides etc. are shown in bold and underlined. N or x=any nucleotide; w=a/t, m=a/c, r=a/g, k=g/t, s=c/g, y=c/t, h=a/t/c, v=a/g/c, d=a/g/t, b=g/t/c. In some cases, specific degeneracy options are indicated in parenthesis: e.g.: (a/g) is either A or G.

SEQ ID NO 1:
R391 int CAG24068.1
MALSVSWLDARLNKEAKETVVKADRDGLSARVSPKGKIVFQFRYRFDGKQ
QRVDIGTYPLMKLAEARNELDRLRAVLDQGRNPKLYLQQERAKYSANQSF
ESIFRDWIDSAGKQGLKEKTWHFQKRSSEIYLLPRLGKYPLTDINELSLR
NCLREVSESSPSNTERLVSVLHKFYDWLIDEQILEINAAAGITAKKVGGK
KGKRTRVLNDTEIRILWRYLHESKITEKNRIYIKLLLLLGGRKGELIQAE
KHHFDLQSAMWTVPIEIRKQGEKIGAPIMRPLIKPAIELIELAMQMSKST
YLFPANGQEELATNGFDTTIPNNVKIWARRSLGVEMEHWSMHDLRRTMRT
RMSAITTQEVAELMIGHSKKGLDAIYNQYQYLDEMRHAYDVWYQQLETII
EPTGFPFNWRFGQ
SEQ ID NO 2:
CAG24069.1 R705
MALSVSWLDARLNKEAKETVVKADRDGLSARASPKGKIVFQFRYRFDGKQ
QRVDIGTYPLMKLAEARNELDRLRAVLDQGRNPKLYLQQERAKYSANQSF
ESIFRDWIDSAGKQGLKEKTWHFQKRSSEIYLLPRLGKYPLTDINELSLR
NCLREVSESSPSNTERLVSVLHKFYDWLIDEQILEINAAAGITAKKVGGK
KGKRTRVLNDNEIRILWRYLHESKITEKNRIYIKLLLLLGGRKGELIQAE
KHHFDLQSAMWTVPIEIRKQGEKIGAPIMQPLIKPAIELIELAMQMSKST
YLFPANGQEELATNGFDTTIPNNVKIWARRSLGVEMEHWSMHDLRRTMRT
RMSAITTQEVAELMIGHSKKGLDAIYNQYQYLDEMRHAYDVWYQQLETII
EPTGFPFNWRFGQ
SEQ ID NO 3:
R997 integrase CAE30340.1
MALSVSWLDARLNKEAKETVVKADRDGLSARVSPKGKIVFQFRYRFDGKQ
QRVDIGTYPLMKLAEARNELDRLRAVLDQGRNPKLYLQQERAKYSANQSF
ESIFRDWIDSAGKQGLKEKTWHYQKRSSEIYLLPRLGKYPLTDINELSLR
NCLREVSESSPSNTERLVSVLHKFYDWLIDEQILEINAAAGITAKKVGGK
KGKRTRVLNDNEIRILWRYLHESKITEKNRIYIKLLLLLGGRKGELIQAE
KHHFDLQSAMWTVPIEIRKQGEKIGAPIMRPLIKPAIELIELAMQMSKST
YLFPANGQEELATNGFDTTIPNNVKIWARRSLSVEMEHWSMHDLRRTMRT
RMSAITTQEVAELMIGHSKKGLDAIYNQYQYLDEMRHAYDVWYQQLZTII
EPTGFPFNWRFGQ
SEQ ID NO 4:
CAE30338.1 pMERPH
MALSVSWLDARLNKEAKETVVKADRDGLSARVSPKGKIVFQFRYRFDGKQ
QRVDIGTYPLMKLAEARNELDRLRAVLDQGRNPKLYLQQERAKYSANQSF
ESIFRDWIDSAGKQGLKEKTWHYQKRSSEIYLLPRLGKYPLTDINELSLR
NCLREVSESSPSNTERLVSVLHKFYDWLIDEQILEINAAAGITAKKVGGK
KGKRTRVLNDNEIRILWRYLHESKITEKNRIYIKLLLLLGGRKGELIQAE
KHHFDLQSAMWTVPIEIRKQGEKIGAPIMRPLIKPAIELIELAMQMSKST
YLFPANGQEELATNGFDTTIPNNVKIWARRSLGIEMEHWSMHDLRRTMRT
RMSAITTQEVAELMIGHSKKGLDAIYNQYQYLDEMRHAYDVWYQQLETII
EPTGFPFNWRFGQ
SEQ ID NO 5:
attL
CGAAGCGCCGCACTTTTGCCATTATTTCTC ACCCTGATGCCGCCCAGCC
T
SEQ ID NO 6:
attR
ATAAGCGAAGGACCTTTGCTATCATCTCTC ACCCGGACGC CGGTAAGA
CT
SEQ ID NO 7
attP
ATAAGCGAAGGACCTTTGCTATCATCTCTC ACCCTGATGCCGCCCAGCC
T
SEQ ID NO 8:
attB
CGAAGCGCCGCACTTTTGCCATTATTT CTCACCCGGACGCCGGTAAGAC
T
SEQ ID No 9:
Orf4
MQMTAVAHQVTPFLTSYEVMARYHISYTTLWRRIKDGSLPQPRINRNTRN
KLWHIEDL EEYEKKED

### REFERENCES

1. Beaber, J W, Hochhut B, Waldor M K (2002) Genomic and functional analyses of SXT, an integrating antibiotic resistance gene transfer element derived from Vibrio cholerae. J Bacteriol 184 (15): 4259-4269.
2. Bernardi, A. and Bernardi, F. (1984) Complete sequence of pSC101. Nucleic Acids Res. 12, 9415-9426.
3. Burrus V, Marrero J and Waldor M K (2006) The current ICE age: Biology and evolution of SXT-related integrating conjugative elements. Plasmid 55(3): 173-183
4. Böltner D, MacMahon C, Pembroke J T, Strike P and Osborn A M (2002) R391 is an 89 kb conjugative genomic island comprising elements related to plasmids, phages, and transposable elements. J. Bacteriol 184:18,5158-5169.
5. Coetzee J N, Datta N & Hedges R W (1972) R factors from Proteus retgeri. J. Gen. Microbiol. 72: 543-552.
6. Ehara M, Nguyen B M, Nguyen D T, Toma C, Higa N & Iwanaga M (2004) Drug susceptibility and its genetic basis in epidemic Vibrio cholerae O1 in Vietnam. Epidem. and Infect. 132 (4): 595-600.
7. Gotoh, T H, and Sekiguchi, M (1977) Mutations to temperature sensitivity in R plasmid pSC101. J. Bacteriol. 131 (2) 405-412.
8. McGrath, B M, and Pembroke, J T (2004) Detailed analysis of the insertion site of the mobile elements R997, pMERPH, R392, R705 and R391 in E. coli K12 FEMS Microbiol Letts 237(1) 19-26.
9. McGrath B M, O'Halloran J A & Pembroke J T (2005) Pre-exposure to UV irradiation increases the transfer frequency of the IncJ conjugative transposon-like elements R391, R392, R705, R706, R997 and pMERPH and is recA+dependent. FEMS Microbiol Letts. 243: 461-465.
10. McGrath B M, O'Halloran J A, Piterina A & Pembroke J T (2006) Molecular probes to detect IncJ elements: a family of integrating, antibiotic resistance mobile genetic elements. J Microbiol Mets. 66 (1): 32-42.
11. McLeod S A, Burrus V, Waldor M K (2006) Requirement for Vibrio cholerae integration host factor in conjugative DNA transfer. J Bacteriol 1881 (6) 5704-5711.
12. Murphy D B, and Pembroke J T (1999) Monitoring of chromosomal insertions of the IncJ elements R391 and R997 in Escherichia coli K12. FEMS Microbiol Letts 174:355-361.
13. Nugent, M E (1981) A conjugative plasmid lacking autonomous replication. J. Gen. Microbiol. 126, 305-310.
14. Nunes-Düby, S E, Kwon H J, Tirumalai R S, Ellenberger T, Landy A (1998) Similarities and differences among 105 members of the Int family of site-specific recombinases. Nucleic Acids Res. 26, 391-406.
15. Osario C R, Marrero J, Wozniak R A F, Lemos M L, Burrus V and Waldor M K (2008) Genomic and functional análisis of ICEPdaSpal, a fish-pathogen-derived SXT-related integrating conjugative element that can mobilise a virulence plasmid. J Bacteriol 190 (9) 3353-3361.
16. Pembroke, J T, Stevens E, Brandsma J A and Van de Putte P (1986) Location and cloning of the UV sensitising function from the chromosomally associated incJ group plasmid R391. Plasmid 16:30-36.
17. Pembroke J T, McGrath B and MacMahon C (2002) The role of conjugative transposons in the Enterobacteriaceae. Cell Mol Lif Sci (CMLS) 59(12) 2055-2064.
18. Pembroke J T and McGrath B M (2005) Pulsed Field Gel Electrophoresis to rapidly detect the presence of IncJ conjugative transposon-like elements. Letts Appl Microbiol. 41: 258-261.
19. Sambrook, J., Fritsch, E. F. & Maniatis, T.; Hrsg. (1989). Molecular Cloning—A Laboratory Manual, 2^{nd} Edition. Cold Spring Habour Laboratory Press, New York.
20. Spada S, Pembroke J T, and Wall J G (2002) Isolation of a novel Thermus thermophilus metal efflux protein that improves E. coli growth under stress conditions. Extremophiles 6:4, 301-308.

1. An integration system comprising
(i) at least one vector capable of expressing at least one exogenous gene, the vector comprising a coding sequence for an attP gene region and a coding sequence for at least one therapeutic molecule; and
(ii) an integrase.

2. An integration system as claimed in claim 1, wherein the integrase is encoded on the at least one vector.

3. An integration system as claimed in claim 1, wherein the integrase is encoded on a second vector capable of expressing at least one exogenous gene.

4. An integration system as claimed in claim 1, wherein the integrase is carried in a liposome.

5. An integration system of any of claims 1 to 4, wherein the integrase has the sequence selected from the group comprising, SEQ ID No. 1, SEQ ID No. 2, SEQ ID No. 3, or SEQ ID No. 4.

6. An integration system of claim 5, wherein the integrase is humanised.

7. An integration system as claimed in claim 1, wherein the at least one vector is temperature sensitive.

8. An integration system as claimed in claim 1, wherein the attP gene region is an attP gene region derived from any member of the SXT/R391 family.

9. An integration system as claimed in claim 8, wherein the attP gene region comprises at least the 17 base pair attP core region together with its upstream promoter and downstream sequence of codons.

10. An integration system as claimed in claim 9, wherein the attP gene region has the sequence of SEQ ID No. 7.

11. An integration system as claimed in claim 1, wherein the first at least one vector further comprises a coding sequence for the bacterial orf4 gene.

12. An integration system as claimed in claim 11, wherein the orf4 gene has the sequence of SEQ ID No. 9.

13. An integration system as claimed in claim 1, wherein the therapeutic molecule is a DNA or RNA molecule.

14. An integration system as claimed in claim 13, wherein the therapeutic molecule is selected from the group comprising NOS (nitric oxide synthase), iNOS (inducible NOS), eNOS (endothelial NOS), VEGF (vascular endothelial growth factor), EFNB2 (Ephrin-B2) and OPN (osteopontin).

15. A method for the delivery of genetic material to a target cell, comprising contacting the cell with an integration system of any of claim 1, 13 or 14.

16. The method of claim 15, wherein the target cell is any cell that contains an attB gene region.

17. A host cell transformed with the integration system of any of claim 1, 13 or 14.

18. A method for the delivery of genetic material to host, comprising administering the integration system of any of claim 1, 13 or 14 to the host.

19. The method of claim 18, wherein the host is suffering from a disease selected from the group comprising cancer, genetic disease, infectious diseases, immunological disorders, neurological disorders or cardiovascular disease wherein the cardiovascular disease includes but is not limited to myocardial infarction, coronary vascular disease, peripheral vascular disease, ischemia, cerebrovascular disease, heart failure.

20. A method of treatment comprising administering to a patient an effective amount of an integration system as claimed in any one of claims 1, 13 or 14.

21. The method of claim 20, wherein the disease is selected from the group comprising cancer, genetic disease, infectious diseases, immunological disorders, neurological disorders or cardiovascular disease wherein the cardiovascular disease includes but is not limited to myocardial infarction, coronary vascular disease, peripheral vascular disease, ischemia, cerebrovascular disease, heart failure.

22. (canceled)

23. A method of treatment comprising administering to a patient an effective amount of a host cell as claimed in claim 17.


SEQUENCE LISTING (28 sequences)

SEQ ID NO: 1 - protein, 413 aa, Artificial Sequence
  R391 int CAG24068.1
MALSVSWLDARLNKEAKETVVKADRDGLSARVSPKGKIVFQFRYRFDGKQQRVDIGTYPLMKLAEARNELDRLRAVLDQGRNPKLYLQQERAKYSANQSFESIFRDWIDSAGKQGLKEKTWHFQKRSSEIYLLPRLGKYPLTDINELSLRNCLREVSESSPSNTERLVSVLHKFYDWLIDEQILEINAAAGITAKKVGGKKGKRTRVLNDTEIRILWRYLHESKITEKNRIYIKLLLLLGGRKGELIQAEKHHFDLQSAMWTVPIEIRKQGEKIGAPIMRPLIKPAIELIELAMQMSKSTYLFPANGQEELATNGFDTTIPNNVKIWARRSLGVEMEHWSMHDLRRTMRTRMSAITTQEVAELMIGHSKKGLDAIYNQYQYLDEMRHAYDVWYQQLETIIEPTGFPFNWRFGQ

SEQ ID NO: 2 - protein, 413 aa, Artificial Sequence
  CAG24069.1  R705
MALSVSWLDARLNKEAKETVVKADRDGLSARASPKGKIVFQFRYRFDGKQQRVDIGTYPLMKLAEARNELDRLRAVLDQGRNPKLYLQQERAKYSANQSFESIFRDWIDSAGKQGLKEKTWHFQKRSSEIYLLPRLGKYPLTDINELSLRNCLREVSESSPSNTERLVSVLHKFYDWLIDEQILEINAAAGITAKKVGGKKGKRTRVLNDNEIRILWRYLHESKITEKNRIYIKLLLLLGGRKGELIQAEKHHFDLQSAMWTVPIEIRKQGEKIGAPIMQPLIKPAIELIELAMQMSKSTYLFPANGQEELATNGFDTTIPNNVKIWARRSLGVEMEHWSMHDLRRTMRTRMSAITTQEVAELMIGHSKKGLDAIYNQYQYLDEMRHAYDVWYQQLETIIEPTGFPFNWRFGQ

SEQ ID NO: 3 - protein, 413 aa, Artificial Sequence
  R997 integrase CAE30340.1
MALSVSWLDARLNKEAKETVVKADRDGLSARVSPKGKIVFQFRYRFDGKQQRVDIGTYPLMKLAEARNELDRLRAVLDQGRNPKLYLQQERAKYSANQSFESIFRDWIDSAGKQGLKEKTWHYQKRSSEIYLLPRLGKYPLTDINELSLRNCLREVSESSPSNTERLVSVLHKFYDWLIDEQILEINAAAGITAKKVGGKKGKRTRVLNDNEIRILWRYLHESKITEKNRIYIKLLLLLGGRKGELIQAEKHHFDLQSAMWTVPIEIRKQGEKIGAPIMRPLIKPAIELIELAMQMSKSTYLFPANGQEELATNGFDTTIPNNVKIWARRSLSVEMEHWSMHDLRRTMRTRMSAITTQEVAELMIGHSKKGLDAIYNQYQYLDEMRHAYDVWYQQLZTIIEPTGFPFNWRFGQ

SEQ ID NO: 4 - protein, 413 aa, Artificial Sequence
  CAE30338.1  pMERPH
MALSVSWLDARLNKEAKETVVKADRDGLSARVSPKGKIVFQFRYRFDGKQQRVDIGTYPLMKLAEARNELDRLRAVLDQGRNPKLYLQQERAKYSANQSFESIFRDWIDSAGKQGLKEKTWHYQKRSSEIYLLPRLGKYPLTDINELSLRNCLREVSESSPSNTERLVSVLHKFYDWLIDEQILEINAAAGITAKKVGGKKGKRTRVLNDNEIRILWRYLHESKITEKNRIYIKLLLLLGGRKGELIQAEKHHFDLQSAMWTVPIEIRKQGEKIGAPIMRPLIKPAIELIELAMQMSKSTYLFPANGQEELATNGFDTTIPNNVKIWARRSLGIEMEHWSMHDLRRTMRTRMSAITTQEVAELMIGHSKKGLDAIYNQYQYLDEMRHAYDVWYQQLETIIEPTGFPFNWRFGQ

SEQ ID NO: 5 - DNA, 50 bp, Artificial Sequence
  attL
cgaagcgccgcacttttgccattatttctcaccctgatgccgcccagcct

SEQ ID NO: 6 - DNA, 50 bp, Artificial Sequence
  attR
ataagcgaaggacctttgctatcatctctcacccggacgccggtaagact

SEQ ID NO: 7 - DNA, 50 bp, Artificial Sequence
  attP
ataagcgaaggacctttgctatcatctctcaccctgatgccgcccagcct

SEQ ID NO: 8 - DNA, 50 bp, Artificial Sequence
  attB
cgaagcgccgcacttttgccattatttctcacccggacgccggtaagact

SEQ ID NO: 9 - protein, 66 aa, Artificial Sequence
  Orf4
MQMTAVAHQVTPFLTSYEVMARYHISYTTLWRRIKDGSLPQPRINRNTRNKLWHIEDLEEYEKKED

SEQ ID NO: 10 - DNA, 31 bp, Artificial Sequence
  Int-For primer
actggatcctagggctgggcttattaacatg

SEQ ID NO: 11 - DNA, 26 bp, Artificial Sequence
  Int -Rev primer
ttagctatacgactctgcagcgaaga

SEQ ID NO: 12 - DNA, 30 bp, Artificial Sequence
  IntHis -For primer
tcaggatccaacatggcgttatcagttagc

SEQ ID NO: 13 - DNA, 30 bp, Artificial Sequence
  IntHis-Rev primer
agcctcgagattttactgtccgaaacgcca

SEQ ID NO: 14 - DNA, 20 bp, Artificial Sequence
  AttP-F-R391 primer
tgatagccgattagataacc

SEQ ID NO: 15 - DNA, 19 bp, Artificial Sequence
  AttP-Rev-R391 primer
acacactttccgaggttac

SEQ ID NO: 16 - DNA, 20 bp, Artificial Sequence
  RT-Int -For primer
caatcagctaatagctggct

SEQ ID NO: 17 - DNA, 20 bp, Artificial Sequence
  RT-Int-Rev primer
accgagatgggctaagtgct

SEQ ID NO: 18 - DNA, 20 bp, Artificial Sequence
  prfCCRE1 primer
tgatagccgattagataacc

SEQ ID NO: 19 - DNA, 22 bp, Artificial Sequence
  prfCCLE1 primer
gtttcttcgttgcacgaactgg

SEQ ID NO: 20 - DNA, 24 bp, Artificial Sequence
  ABF1 (For100) primer
cttatttgcaagaagtggcgaagc

SEQ ID NO: 21 - DNA, 21 bp, Artificial Sequence
  ABF2 (For150) primer
cgatgccgcttactcaagaag

SEQ ID NO: 22 - DNA, 22 bp, Artificial Sequence
  ABF3 (LE1) primer
gtttcttcgttgcacgaactgg

SEQ ID NO: 23 - DNA, 23 bp, Artificial Sequence
  ABF4 (For500) primer
cgatattctcggtggcatcaacg

SEQ ID NO: 24 - DNA, 22 bp, Artificial Sequence
  ABR1 (RE1) primer
cggtctgaatggcctgtccgaa

SEQ ID NO: 25 - DNA, 22 bp, Artificial Sequence
  ABR2 (Rev300) primer
cgacttagcgtgctggtggaac

SEQ ID NO: 26 - DNA, 22 bp, Artificial Sequence
  ABR3 (Rev400) primer
ggtgtcgagcaggttaaccagg

SEQ ID NO: 27 - DNA, 19 bp, Artificial Sequence
  ABR4 (Rev500) primer
gccgtcaggtacgatagga

SEQ ID NO: 28 - DNA, 17 bp, Artificial Sequence
  att 391
atcatctcncaccctga